(19) United States
(12) Patent Application Publication
(10) Pub. No.: US 2004/0091435 A1
(43) Pub. Date: May 13, 2004

(54) Deodorant and antiperspirant controlled release system

(76) Inventors: Adi Shefer, Dayton, NJ (US); Samuel David Shefer, Dayton, NJ (US)

Correspondence Address: Diane Dunn McKay, Esq., Mathews, Collins, Shepherd & McKay, P.A., Suite 306, 100 Thanet Circle, Princeton, NJ, 08540, US

(21) Appl. No.: 10/293,574

(22) Filed: Nov. 13, 2002

(51) Int. Cl. A61K 7/32

(52) U.S. Cl. 424/65

(57) ABSTRACT

The present invention relates to a controlled release system for deodorant and antiperspirant aqueous gels comprising active ingredients and sensory markers encapsulated in solid hydrophobic micro spheres to enhance the stability of the active ingredients and sensory markers in the product base. The present invention provides long lasting fragrance residue and deodorancy on the skin, over an extended period of time. A wide range of active ingredients and sensory markers can be used in the present invention.

### BACKGROUND OF THE INVENTION

1. Field of the Invention

The present invention relates to underarm products that contain water, such as deodorant and antiperspirant gels, comprising active ingredients as well as sensory markers encapsulated in hydrophobic micro spheres.

2. Description of the Related Art

The cosmetic industry has searched many years for ways to enhance the performance of consumer and cosmetic products and make them more aesthetically pleasing for the consumers. Consumer acceptance of cosmetic products is determined not only by the performance achieved with these products but also the aesthetics associated therewith. Fragrances are an important aspect of the successful products and they are being utilized, in addition to imparting an aesthetically pleasing odor, as sensory markers to convey to the consumer the product performance and effectiveness.

Consumers are becoming increasingly educated and expect a high level of sophistication in their products and the market has become extremely competitive. Many consumers would prefer for the fragrance present in these products, to last longer or be released only upon need.

Extensive work has been done in recent years to create enduring fragrances that have increased fragrance longevity on various surfaces such as fabric, skin, and hair through the careful selection of fragrance ingredients that have specific chemical and physical properties (see U.S. Pat. Nos. 6,184,188, 6,147,037, 6,086,903, 5,919,752, 5,849,310, 5,833,999, 5,830,835, 5,652,206 5,562,847, 5,540,853, 5,531,910, 5,500,154, 5,500,138, and 5,500,137, incorporated herein by reference).

Attempts have been made to fulfill the foregoing needs for fragrance longevity in underarm products that contain water by encapsulating the fragrance in plastic materials and polymers. U.S. Pat. No. 4,217,426 discloses a semi-crystalline polyester/low viscosity polyethylene melt blend, which is non-tacky and non-blocking and readily grindable by means of cryogenic grinding techniques, for providing powders suitable for powder adhesives. The powders are particularly useful for fusible interlinings or for providing powder coating materials.

U.S. Pat. Nos. 4,731,243 and 4,934,609 disclose deodorant and/or antiperspirant vehicles which contain fragrance-containing polymer pellets containing 1 to 80% fragrance. The polymeric pellets are produced by means of cryogenically grinding an extruded mixture of perfume composition and polymer. The drawback of fragrance controlled release system disclosed in U.S. Pat. Nos. 4,731,243 and 4,934,609 is that production of these particles, consists of a two step process (i.e., extrusion and grinding) which makes the production of the fragrance-particles to have high manufacturing costs. In addition cryogenically grinding of the extruded mixture creates particles of irregular shape and size, disrupts the material structure, and affects morphology. Cryogenically grinding also exposes a large surface area of the particles to the atmosphere and enhances diffusion and evaporation of the fragrance from the large exposed surface area.

It is desirable to provide a fragrance release system with improved product shelf life and to provide fragrance longevity or long lasting deodorancy on skin over an extended period of time from deodorant or antiperspirant products.

### SUMMARY OF THE INVENTION

The present invention addresses the ongoing need for fragrance controlled release systems that can be incorporated into aqueous underarm products, such as, deodorant and antiperspirant aqueous gels, to encapsulate active ingredients and sensory markers. The fragrance controlled release system of the present invention enhances the stability of a wide range of active ingredients and sensory markers in the product base as well as provides long lasting fragrance residue and deodorancy on the skin.

The present invention also provides a free-flowing powder comprising micro-spheres composed of waxy materials that encapsulate active ingredients and sensory markers characterized by:

(i) protection of the active ingredients and sensory markers during storage, until needed; and

(ii) long lasting residue of active ingredients and sensory markers on the skin

The present invention further provides aqueous underarm products such as deodorant gels, antiperspirant gels, deodorant or antiperspirant roll on devices, deodorant or antiperspirant smooth ons, deodorant or antiperspirant aerosols, and the like comprising the micro spheres of the present invention.

The micro spheres are in the form of free flowing powder produced by spraying processes such as spray chilling, spray congealing, granulation, and the like, to create fine or very fine particles. The particles typically have a substantially spherical shape. The particles have an average particle diameter of less than about 1 mm.

### DETAILED DESCRIPTION

The present invention relates to a controlled release system for deodorant and antiperspirant aqueous gels comprising active ingredients and sensory markers encapsulated in solid hydrophobic micro spheres to enhance the stability of the active ingredients and sensory markers in the product base. The present invention provides long lasting fragrance residue and deodorancy on the skin, over an extended period of time. For example the extended period of time can be in the range of about 12 hours to about 24 hours. A wide range of active ingredients and sensory markers can be used in the present invention.

The invention also provides wax micro-spheres in the form of free-flowing powder that encapsulate active ingredients and sensory markers characterized by:

(i) protection of the active ingredients and sensory markers during storage, until needed; and

(ii) long lasting residue of active ingredients and sensory markers on skin

The invention further provides aqueous underarm products such as deodorant gels, antiperspirant gels, deodorant or antiperspirant roll on devices, deodorant or antiperspirant smooth ons, deodorant or antiperspirant aerosols, and the like comprising the wax micro spheres of the present invention. The micro spheres are in the form of free flowing powder. The microspheres have an average diameter of less than about 1 mm. The wax micro spheres are produced by means of atomizing a melt using spray congealing, spray chilling, or granulation. The term “spheres” is intended to describe solid, substantially spherical particulates. It is appreciated that other particle shapes can be included in the term “sphere” in accordance with the teachings of the present invention.

Matrix Materials for Forming the Microspheres

Suitable solid core materials for forming microspheres of the present invention are inert nontoxic natural, regenerated, or synthetic waxes including animal waxes such as beeswax, lanolin and shellac wax, vegetable waxes such as carnauba, candelilla, sugar cane, rice bran, and bayberry wax, mineral waxes such as petroleum waxes including paraffin and microcrystalline wax, ozokrite wax, and mixtures thereof. Other hydrophobic materials which can be used in the present invention include wax and silicon copolymers, such as candelilla wax and silicone copolymer, ozokrite wax and silicon copolymers, beeswax and silicon copolymers, and the like. Other hydrophobic compounds which can be used in the present invention include: fatty acid esters such as cetyl palmitate, ethyl stearate, isopropyl myristate, and isopropyl palmitate; high molecular weight fatty alcohols such as cetostearyl alcohol, cetyl alcohol, stearyl alcohol, and oleyl alcohol, solid hydrogenated castor and vegetable oils, hard paraffins, hard fats, and mixtures thereof. Other hydrophobic compounds which can be used, include triglycerides, preferably of at least food grade purity, which can be produced by synthesis or by isolation from natural sources. Natural sources can include animal fat or vegetable oil, such as soy oil, as a source of long chain triglycerides (LCT). Other triglycerides suitable for use in the present invention are composed of a majority of medium length fatty acids (C10-C18), denoted medium chain triglycerides (MCT). The fatty acid moieties of such triglycerides can be unsaturated or polyunsaturated and mixtures of triglycerides having various fatty acid material. The micro particle matrix can comprise a single wax material or a mixture of a plurality of materials. Other waxy materials that are known to those skilled in the art and suitable materials as described in “Industrial Waxes,” Vol. I and II, by Bennett F.A.I.C., published by Chemical Publishing Company Inc., 1975 and Martindale, “The Extra Pharmacopoeia”, The Pharmaceutical Press, 28^{th} Edition pp. 1063-1072, 1982.

Active Agents

The controlled release system of the present invention includes a wide range of cosmetic, dermatological, and pharmaceutical active agents, including, but are not limited to: anti-oxidants; free radical scavengers; moisturizers; humectants; antimicrobial (e.g., antibacterial) agents; allergy inhibitors; anti-inflammatory agents; fresheners; healing agents; deodorants and antiperspirants agents; skin emollients and skin moisturizers; vitamins; fragrances; herbal extracts; cooling agents; heating agents; skin conditioners; coloring agents and dyes; moisture absorbers; sebum absorbers; skin penetration enhancers; and the like; and other active ingredients.

Vitamins

Various vitamins can be included in the release system of the present invention. For example, vitamin A and derivatives thereof, vitamin B₂, biotin, pantothenic acid, vitamin K, vitamin D, vitamin E and mixtures thereof can be used.

Skin Conditioners

The micro spheres can also contain skin conditioners, and moisturizers. Suitable conditioners include mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Suitable moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

Fragrances, Flavors, and Sensory Markers

The micro spheres of the present invention can also contain sensory markers such as fragrances, cooling agents and heating agents. For example, the cooling agents can be menthol derivatives and the heating agents can be capzasin. The release of the sensory markers from the microspheres can be used to convey to the consumer the product performance, provide long lasting odor or flavor perception, and signal that a new application of the product is needed. Conventional fragrance ingredients and perfume ingredients can be used in the controlled release system of the present invention. Selection of any perfume component, or amount of perfume, is based on functional and aesthetic considerations. Examples of usable fragrance and flavor compounds discussed hereinafter, along with their odor characters, and their physical and chemical properties, are given in “Perfume and Flavor Chemicals (Aroma Chemicals)”, Steffen Arctander, published by the author, 1969, and in “Common Fragrance and flavor Materials—Preparation, Properties and Uses”, Kurt Bauer and Dorotea Garbe, published by VCH Verlagsgesellschaft mbH, 1985, incorporated herein by reference.

Botanical extracts can be used in the controlled release system including oak bark extract, walnut extract, tincture of arnica, hamamelis extract, ribwort extract, pansy extract, thyme or sage extract. Materials can be included in the controlled release system of the present invention for the treatment of damaged or injured skin, for example, St. John's wort tincture, cone flowers tincture, chamomile flowers extract, or calendula flowers tincture. Materials can be included in the controlled release system of the present invention for the care of exhausted and damaged skin, for example, birch leaves extract, nettle extract, coldsfoot extract, comfrey tincture, horsetail extract, or aloe vera extract. Vegetable preparations can also be released from the controlled release system of the present invention for the intradermal treatment of diseases, for example, extracts of horse chestnut and butcher's broom in case of vein diseases, or extracts and tinctures of arnica, calendula, and capsicum in case of contusions, distortions, or haemorrhages. Vegetable preparations can be used in the controlled release system according to the present invention for transdermal therapy, for example, ginseng extract in case of geriatric complaints; valerian tincture, extracts of melissa and hop to cause a sedative effect in case of superexcitation, sleep disturbances, and stress; extracts of kola and tea to achieve a stimulative effect; or hawthorn extract to stabilize the circulatory system.

Preservatives

Preservatives can desirably be incorporated into the controlled release system of the present invention to protect against the growth of potentially harmful microorganisms. While microorganisms tend to grow in the aqueous phase, microorganisms can also reside in the anhydrous or oil phase. As such, preservatives which have solubility in both water and oil are preferably employed in the present compositions. Suitable preservatives for compositions of the present invention are alkyl esters of parahydroxybenzoic acid. Other preservatives, which can be used include hydantoin derivatives, propionate salts, and a variety of quaternary ammonium compounds.

Appropriate preservatives can be selected to satisfy the preservative challenge test and to provide product stability. Particularly preferred preservatives are methylparaben, imidazolidinyl urea, sodium dehydroacetate, propylparaben, trisodium ethylenediamine tetraacetate (EDTA), and benzyl alcohol. The preservative can be selected based on the consideration of possible incompatibilities between the preservative and other ingredients in the controlled release system. Preservatives are preferably employed in amounts ranging from about 0.01% to about 2% by weight of the composition of the microsphere.

Dyes & Pigments

The wax micro spheres of the present invention may include colorants such as natural or synthetic dyes and pigments, for example FD&C Yellow #10, Eyeshadow Blue KO, Colour Index 77 510, EG-No., Blue 15 (C-Blue 17), or mixtures of dyes and pigments Eyeshadow Blue KO and Lemon Yellow ZN 3, F.D. & C. Blue No. 1 Lake, F.D. & C. Blue No. 2 Lake, F.D. & C. Red No. 3 Lake, F.D. & C. Yellow No. 5 Lake and F.D. & C. Yellow No. 6 Lake.

Pigments suitable for use herein are all inorganic and organic colors/pigments suitable for use in deodorant compositions. These are usually aluminum, barium or calcium salts or lakes. Examples of lakes that can be used are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake. Other colors and pigments can also be included in the lip compositions, such as pearls, titanium oxides, Red 6, Red 21, Blue 1, Orange 5, and Green 5 dyes, chalk, talc, iron oxides and titanated micas.

Gel Formulation

A clear gel antiperspirant is set forth in International Patent Application No. WO 92/05767, published on Apr. 16, 1992 (The Gillette Company), which is incorporated herein by reference. This patent application pertains generally to a clear gel-type cosmetic product which includes an emulsion with an oil phase and a water phase that includes an incorporated active ingredient. The oil phase preferably makes up about 10 to 25% of the product and includes an emulsifier which when properly mixed with the water phase components yields a water-in-oil emulsion. The oil phase is typically a blend of liquids and includes a polyorganosiloxane (e.g., dimethicone) and a silicone emulsifying agent. A particularly suitable emulsifying agent is a polyether substituted silicone of cyclomethicone and dimethicone copolyol. This emulsifier is useful for preparing stable water-in-oil silicone emulsions where silicone makes up a large portion of the oil phase, and is a dispersion of a silicone surfactant (i.e., dimethicone copolyol), i.e., 10% silicone surfactant in cyclomethicone (i.e., a silicone solvent). The water phase includes one or more polar species such as water, propylene glycol, sorbitol and ethanol. The water phase includes, in solution, a deodorant and/or antiperspirant active ingredient such a triclosan, benzethonium chloride and/or an astringent salt of aluminum or zirconium, such as aluminum chlorohydrate or aluminum zirconium tetrachlorohydrex-glycine. The gel can also contain additional cosmetic ingredients such as emollients, colorants, fragrances, and preservatives.

International Patent Application No. WO 97/06777, which is incorporated herein by reference, also discloses a clear cosmetic gel composition which includes: (1) an aqueous phase containing water and at least one cosmetically active ingredient, (2) an oil phase containing a high refractive index material, (3) at least one coupling agent to bring the aqueous phase and the oil phase into a homogeneous composition, and (4) an alkoxylated, alkyl substituted siloxane surface active agent in an amount sufficient to form the composition into a water-in-oil emulsion. The oil phase includes a volatile silicone fluid, a non-volatile silicone fluid and an emollient. The emollient is preferably phenyl trimethicone.

U.S. Pat. No. 4,900,542 (Parrotta, Jr., et al.), which issued on Feb. 13, 1990 and which is incorporated herein by reference, discloses a process for preparing uniform, clear, microcrystalline emulsion antiperspirant compositions of gel-like consistency comprising: mixing the antiperspirant active material with water, charging the aqueous phase into an oil-alcohol phase containing a volatile silicone, a silicone emulsifier, a non-volatile emollient and a coupling agent, heating the resultant mixture with agitation until a uniform mixture is obtained, homogenizing the mixture and passing the homogenized mixture to a holding tank or directly to a filter.

Another clear cosmetic gel composition can comprise: (a) an aqueous phase comprising: (i) water, and (ii) at least one cosmetically active ingredient; (b) a coupling agent; (c) an oil phase comprising: (i) a silicone-containing solvent, and (ii) an isoparaffin solvent having a boiling range between about 100 to 340. degree. C., wherein the isoparaffin constitutes between about 1 to 75% by weight, of the total of the oil phase; and (d) silicone-containing surfactant, such as that disclosed in U.S. Pat. No. 6,447,791 incorporated herein as reference.

Other underarm gel compositions are disclosed in U.S. Pat. No. 6,007,799, incorporated herein as reference. The patent disclosed is a clear cosmetic gel composition in the form of a water-in-oil emulsion, and methods of forming and of using the composition. The composition has a water-based phase containing water, a cosmetically active ingredient, and at least one coupling agent; and an oil-based phase containing a material having a refractive index in the range of 1.40-1.50, silicone fluids and an alkoxylated, alkyl substituted siloxane surface active agent (e.g., dimethicone copolyol). The composition has a refractive index in a range of 1.4026 to 1.4150. Where the cosmetically active ingredient is an antiperspirant active ingredient, the composition can be an antiperspirant gel (e.g., soft gel) composition. In the refractive index range of the present invention, increased amounts of, e.g., antiperspirant active ingredient, and other high-refractive-index materials providing cosmetic benefits, can be incorporated in the water and oil phases of the composition while still achieving a clear composition. The composition can also include polypropylene glycols (e.g., tripropylene glycol), as part of the water-based phase, to provide a composition having reduced tackiness and reduced whitening (decreased residue); this composition is also mild (reduced skin irritation potential) relative to comparable commercial products.

Processing Method

The micro spheres of the present invention can be prepared by co-melting the sensory markers and/or other active ingredients with the wax materials and then converting the molten mass into spheres of the desired size by spraying the mass through a nozzle into a cool atmosphere. Particle size selection can be accomplished by screening, air stream segregation, and the like.

The process for producing the micro spheres comprises the following steps:

(i) heating the matrix material, such as wax or solid hydrophobic materials to about 10 degrees above the melting point of the ingredients, with continuous agitation;

(ii) adding selected active ingredients to the melt with continuous agitation; and

(iii) cooling the melt to ambient temperature to form a dry free-flowing powder composition.

The molten mixture can be converted into a free-flowing powder by spraying processes known in the art, such as spray chilling, spray-congealing, drum chilling, granulation, and the like to create fine or very fine particles, of a substantially spherical shape, having an average particle diameter between about 1 micron and about 1 millimeter.

Spraying processes are particularly suitable in which the melts are converted into fine or very fine particles, primarily of spherical shape, whilst they are finely divided and in free fall. The spraying processes can be assisted by blowing with countercurrent cold air such as by spray-chilling, spray-congealing.

A flow agent is preferably added after the powder is manufactured. Flow agents which can be used in the present invention can be silica, clay, starch, and the like which can be added to the particles. Suitable fine silica materials are commercially available as pyrogenic or fumed silicas, such as materials sold under Trade names of Cabosil manufactured by G. L. Cabot Inc., Aerogel 500 manufactured by J. M. Huber Corp., Syloid 244, -63, -65 manufactured by W. R. Grace and Co., Li-sil 233 manufactured by Pittsburg Plate Glass Co., and Sipernat D-17 manufactured by Degussa Co. Suitable clay materials include kaolinites and bentonites, as described in British Pat. No. 1,460,646.

Spray chilling, or spray congealing is well known in the art and been used commercially in many applications, including foods where the core material is a flavoring oil and cosmetics where the core material is a fragrance oil, see “Flavor Encapsulation”, edited by Risch S. J. and Reineccius G. A., ACS Symposium Series, 1988; “Multiparticulate Oral Drug Delivery” pp.17-34, edited by Ghebre-Sellassie I., Drugs and the Pharmaceutical Sciences, Vol. 65, 1994 which are incorporated herein as references.

The processing method described herein is simple and economical and is characterized by high loading, reproducibility, versatility, and stability. The method is further illustrated in the non-limiting examples.

Active ingredients and fragrances may diffuse from the particles at any of the rates of the following:

(i) at steady-state or zero-order release rate in which there is substantially continuous release per unit of time;

(ii) a first-order release rate in which the rate of release declines toward zero with time; and

(iii) a delayed release in which the initial rate is slow, but then increases with time.

(iv) The active agent contained in the particles can be released an extended period of time up to a period of few days to few weeks, depending on matrix barrier properties, particle size, and active payload.

Micro spheres formed of a hydrophobic material provide a controlled release system in order to release the active agent over an extended period of time by molecular diffusion. Active agents in the hydrophobic matrix of the particles can be released by transient diffusion. The theoretical early and late time approximation of the release rate active ingredients and fragrances dissolved in the hydrophobic matrix of the spheres can be calculated from the following equations:

Early Time Approximation

$$\begin{matrix}( {m}_{t} / {m}_{sec} ) < 0.4 & \\ \frac{{M}_{t}}{{M}_{∞}} = 4 {( \frac{{D}_{p} t}{Π {r}^{2}} )}^{1 / 2} - \frac{{D}_{p} t}{{r}^{2}} & ( 1 ) \\ \frac{ⅆ {M}_{t} / {M}_{∞}}{ⅆ t} = 2 {( \frac{{D}_{p}}{Π {r}^{2} t} )}^{1 / 2} - \frac{{D}_{p}}{{r}^{2}} & ( 2 )\end{matrix}$$

Late Time Approximation

$$\begin{matrix}( {m}_{t} / {m}_{∞} ) > 0.6 & \\ \frac{{M}_{t}}{{M}_{∞}} = 1 - \frac{4}{{( 2.405 )}^{2}} exp ( \frac{- {( 2.405 )}^{2} {D}_{p} t}{{r}^{2}} ) & ( 3 ) \\ \frac{ⅆ {M}_{t} / {M}_{∞}}{ⅆ t} = 1 - \frac{4 {D}_{p}}{{r}^{2}} exp ( \frac{- {( 2.405 )}^{2} {D}_{p} t}{{r}^{2}} ) & ( 4 )\end{matrix}$$

wherein:

r is the radius of the cylinder,

m ∞ is the amount fragrance released from the controlled release system after infinite time;

mₜ is the amount fragrance released from the controlled release system after time t; and

Dₚ is the diffusion coefficient of the fragrance or aroma chemical in the matrix.

The invention can be further illustrated by the following examples preferred embodiments thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### Preparation of Fragrance Wax Micro Spheres

### EXAMPLE 1

Wax micro spheres for underarm applications comprising a fragrance were prepared using a fresh fragrance obtained from Noville Inc. The wax matrix material is candelilla wax commercially available from Strahl & Pitsch Inc.

500 grams of candelilla wax has been placed in the oven at 90° C. 500 grams of the fresh fragrance was added to the molten wax while mixing it with a propeller mixer. This molten solution is atomized into a chamber with ambient temperature air passing through the chamber. The atomized droplets freeze into solid particles in the size range of about 800 microns.

### EXAMPLE 2

Wax micro spheres for underarm applications comprising a fragrance were prepared using a fresh fragrance obtained from Noville Inc. The wax matrix material is Ozokerite wax commercially available from Strahl & Pitsch Inc.

500 grams of ozokerite wax has been placed in the oven at 90° C. 500 grams of the fresh fragrance was added to the molten wax while mixing it with a propeller mixer. This molten solution is atomized into a chamber with ambient temperature air passing through the chamber. The atomized droplets freeze into solid particles in the size range of about 500 microns.

### Preparation of Colored Wax Micro Spheres

### EXAMPLE 3

Wax micro spheres for underarm applications comprising a colorant were prepared using a Green #5. The wax matrix material is a mixture of ozokerite wax commercially available from Strahl & Pitsch Inc and glyceryl monostearate, commercially available from Jeen International of Fairfield New-Jersey under the trade name Jeechem GMS-450.

499 grams of candelilla wax and 499 grams of glyceryl monostearate have been placed in the oven at 90° C. 2 grams of the colorant green #5 was added to the molten wax while mixing it with a propeller mixer. This molten solution is atomized into a chamber with ambient temperature air passing through the chamber. The atomized droplets freeze into solid particles in the size range of about 800 microns.

### Incorporation of Wax Micro Spheres in Underarm Products

### EXAMPLE 4

The wax micro sphere of Example 1 were incorporated in an antiperspirant gel formulation (Happi Magazine Formulary April 2000).

Antiperspirant Gel Furmulation

| | |
|---|---|
| Ingredients: | % Wt. |

Phase A

| | |
|---|---|
| Abil EM 97 (Goldschmidt) (dimethicone copolyol (and) | 2.40 |
| cyclopentasiloxane) | |
| Abil B 8839 (Goldschmidt) (cyclopentasiloxane (and) | 13.80 |
| cyclohexasiloxane) | |
| Tegosoft (Goldschmidt) (isopropyl palmitate) | 0.50 |
| Dimethicone | 0.80 |
| Abil AV 20 (Goldschmidt) (phenyl trimethicone) | 0.50 |
| Abil B 8832 (Goldschmidt) (dimethicone copolyol) | 0.80 |
| Tegosoft SH (Goldschmidt) (stearyl heptanoate) | 0.75 |
| Phase B | |
| Aluminum chlorohydrate (50% solution) | 50.00 |
| SD Alcohol 40 | 4.00 |
| Sorbitol | 0.50 |
| Propylene glycol | 13.95 |
| Wax micro spheres of Example 1 or 2 gr. of neat fragrance | 4.00 |
| Water | 5.00 |
| Dipropylene glycol | 3.00 |
| Preservative | q.s. |

Procedure

Combine phase A ingredients, mixing to uniformity at room temperature. Add phase B ingredients in separate container, mixing to uniformity. The active salt should be mixed to a clear colorless solution. Measure the refractive indices of both phases. Adjust phase B using propylene glycol to raise, or water to lower, the refractive index of phase B to match that of phase A. The refractive indices should agree to the fourth decimal place for total clarity. Slowly stream phase B into phase A with slow (300 rpm) multiblade mixing. The addition rate should match the agitation, not allowing the water to pool on the emulsion surface. After the addition of the water phase is complete, increase the agitation rate to 1,200 rpm for a few minutes. This will build the viscosity of the mixture to a low viscosity flowing gel. Homogenize the mixture at a low rate. Mix until a firm gel is obtained. The wax micro spheres are added to the final product with slow mixing.

### EXAMPLE 5

The wax micro sphere of Example 2 and Example 3 were incorporated in an antiperspirant gel formulation (Happi Magazine Formulary April 2000).

Antiperspirant Gel Furmulation

| | |
|---|---|
| Ingredients: | % Wt. |

Phase A

| | |
|---|---|
| Abil EM 97 (Goldschmidt) (dimethicone copolyol (and) | 2.40 |
| cyclopentasiloxane) | |
| Abil B 8839 (Goldschmidt) (cyclopentasiloxane (and) | 13.80 |
| cyclohexasiloxane) | |
| Tegosoft (Goldschmidt) (isopropyl palmitate) | 0.50 |
| Dimethicone | 0.80 |
| Abil AV 20 (Goldschmidt) (phenyl trimethicone) | 0.50 |
| Abil B 8832 (Goldschmidt) (dimethicone copolyol) | 0.80 |
| Tegosoft SH (Goldschmidt) (stearyl heptanoate) | 0.75 |
| Phase B | |
| Aluminum chlorohydrate (50% solution) | 50.00 |
| SD Alcohol 40 | 4.00 |
| Sorbitol | 0.50 |
| Propylene glycol | 13.95 |
| Wax micro spheres of Example 2 or 2 gr. of neat fragrance | 4.00 |
| Water | 5.00 |
| Colored wax micro spheres of Example 3 | 3.00 |
| Preservative | q.s. |

Procedure

Combine phase A ingredients, mixing to uniformity at room temperature. Add phase B ingredients in separate container, mixing to uniformity. The active salt should be mixed to a clear colorless solution. Measure the refractive indices of both phases. Adjust phase B using propylene glycol to raise, or water to lower, the refractive index of phase B to match that of phase A. The refractive indices should agree to the fourth decimal place for total clarity. Slowly stream phase B into phase A with slow (300 rpm) multiblade mixing. The addition rate should match the agitation, not allowing the water to pool on the emulsion surface. After the addition of the water phase is complete, increase the agitation rate to 1,200 rpm for a few minutes. This will build the viscosity of the mixture to a low viscosity flowing gel. Homogenize the mixture at a low rate. Mix until a firm gel is obtained. The wax micro spheres are added to the final product with slow mixing.

### Evaluation of Product Performance—Long Lasting Deodorancy

### EXAMPLE 6

The antiperspirant samples were applied on the forearm. The ability of the products to provide long lasting fragrance residue on skin was determined by sensory evaluation 4 hours and 8 hours after application of the product. At all evaluation points, the skin area treated with the product comprising the fragranced wax micro spheres of the present invention were found to provide higher odor intensity compared to the control sample comprising the neat. Odor perception is, by its nature, a very subjective determination. According to the procedure, the samples to be tested are provided to a panel of six odor specialists who independently rank the odor intensity on a scale of 1 (least) to 10 (most) for odor and intensity. Samples yielding an odor ranking below about 3.0 possess an odor which would hardly be noticed by the general public. The odor evaluation results were as follows:

| | Odor intensity | |
|---|---|---|
| | Evaluated after 4 Hours | Evaluated after 8 Hours |
| Neat Fragrance Oil | 4 | 2 |
| Encapsulated Fragrance (Example 1) | 7 | 5 |

These results clearly show that only the forearm treated with the fragrance encapsulated in the wax micro spheres of Example 1 provide long lasting fragrance residue on skin that can be easily perceived. The forearm treated with the neat fragrance had very low intensity.

What is claimed is:

1. A controlled delivery system for a deodorant or antiperspirant comprising:
a plurality of solid microspheres, each of said solid microspheres comprising an effective amount of an agent encapsulated in a waxy material.

2. The system of claim 1 wherein said waxy material is selected from one or more of the group consisting of natural wax, regenerated wax, synthetic wax, animal wax, vegetable wax, mineral wax, natural wax and silicon copolymer, synthetic wax and silicon copolymer, fatty acid ester, fatty alcohol, vegetable oil, hard paraffin, hard fat, triglyceride, solid hydrogenated plant oil, hydrogenated castor oil and hydrogenated vegetable oil.

3. The system of claim 1 wherein said active agent is one or more of a fragrance, cosmetic agent, dermatological agent or pharmaceutical agent.

4. The system of claim 1 wherein said active agent comprises one or more agents selected from the group consisting of: anti-oxidants; free radical scavengers; moisturizers; humectants; antimicrobial agents; antibacterial agents; allergy inhibitors; anti-inflammatory agents; fresheners; healing agents; deodorants; antiperspirants; skin emollients; skin moisturizers; vitamins; fragrances; herbal extracts; cooling agents; heating agents; skin conditioners; coloring agents; coloring dyes; moisture absorbers; sebum absorbers; and skin penetration enhancers.

5. The system of claim 1 wherein said active agent is a fragrance and one or more of a heating agent or cooling agent.

6. The system according to claim 1 wherein said active agent is released for an extended period of time.

7. The system according to claim 6 wherein the extended period of time is in the range of about 12 to about 24 hours.

8. The system of claim 1 wherein said microsphere has of about 1.0 micron to about 1.0 mm.

9. The system of claim 1 further comprising a deodorant or antiperspirant.

10. The composition of claim 9 wherein the desired form of said deodorant or antiperspirant is chosen from a gel, cream, stick form and aerosol.

11. A gel composition for a deodorant or antiperspirant comprising:
a plurality of solid microspheres, each of said solid microspheres comprising an effective amount of an agent encapsulated in a waxy material,
wherein the composition is applied to skin.

12. The composition of claim 11 wherein said waxy material is selected from one or more of the group consisting of natural wax, regenerated wax, synthetic wax, animal wax, vegetable wax, mineral wax, natural wax and silicon copolymer, synthetic wax and silicon copolymer, fatty acid ester, fatty alcohol, vegetable oil, hard paraffin, hard fat, triglyceride, solid hydrogenated plant oil, hydrogenated castor oil and hydrogenated vegetable oil.

13. The composition of claim 11 wherein said active agent is one or more of a fragrance, cosmetic agent, dermatological agent or pharmaceutical agent.

14. The composition of claim 11 wherein said active agent comprises one or more agents selected from the group consisting of: anti-oxidants; free radical scavengers; moisturizers; humectants; antimicrobial agents; antibacterial agents; allergy inhibitors; anti-inflammatory agents; fresheners; healing agents; deodorants; antiperspirants; skin emollients; skin moisturizers; vitamins; fragrances; herbal extracts; cooling agents; heating agents; skin conditioners; coloring agents; coloring dyes; moisture absorbers; sebum absorbers; and skin penetration enhancers.

15. The composition of claim 11 wherein said active agent is a fragrance and one or more of a heating agent or cooling agent.

16. The composition of claim 11 further comprising deodorant or antiperspirant.

17. The composition of claim 16 wherein the desired form of said deodorant or antiperspirant is chosen from a gel, cream, stick form and aerosols.

18. An article of manufacture comprising said system of claim 11.

19. The article of claim 18 wherein said article is selected from the group consisting of deodorant gel, antiperspirant gel, deodorant roll on, antiperspirant roll on, deodorant smooth on, antiperspirant smooth on, deodorant aerosol and antiperspirant aerosol.

20. A solid microsphere comprising:
a waxy material encapsulating an active agent,
wherein said microsphere is applied on skin.

21. The microsphere of claim 20 wherein said hydrophobic material is selected from one or more of the group consisting of natural wax, regenerated wax, synthetic wax, animal wax, vegetable wax, mineral wax, natural wax and silicon copolymer, synthetic wax and silicon copolymer, fatty acid ester, fatty alcohol, vegetable oil, hard paraffin, hard fat, triglyceride, solid hydrogenated plant oil, hydrogenated castor oil and hydrogenated vegetable oil.

22. The system of claim 20 wherein said active agent is one or more of a fragrance, cosmetic agent, dermatological agent or pharmaceutical agent.

23. The system of claim 20 wherein said active agent comprises one or more agents selected from the group consisting of: anti-oxidants; free radical scavengers; moisturizers; humectants; antimicrobial agents; antibacterial agents; allergy inhibitors; anti-inflammatory agents; fresheners; healing agents; deodorants; antiperspirants; skin emollients; skin moisturizers; vitamins; fragrances; herbal extracts; cooling agents; heating agents; skin conditioners; coloring agents; coloring dyes; moisture absorbers; sebum absorbers; and skin penetration enhancers.

24. The system of claim 20 wherein said active agent is a fragrance and one or more of a heating agent or cooling agent.

25. A method of treating human odor comprising applying to skin an effective amount of a composition comprising:
a plurality of solid microspheres, each of said solid microspheres comprising an effective amount of an agent encapsulated in a waxy material.

26. The method of claim 25 wherein said active agent is one or more of a fragrance, cosmetic agent, dermatological agent or pharmaceutical agent.

27. A method of enhancing the effectiveness of deodorant or antiperspirant composition comprising the step of adding to said composition a composition comprising a plurality of solid microspheres, each of said solid microspheres comprising an effective amount of an agent encapsulated in a waxy material.

28. The method of claim 27 wherein said active agent is one or more of a fragrance, cosmetic agent, dermatological agent or pharmaceutical agent.

29. A method for forming the system of claim 1 comprising the steps of:
heating the waxy material to about 10 degrees above the melting point with continuous agitation;
adding said active agent to the melt with continuous agitation; and
cooling the melt to ambient temperature to form a dry free-flowing powder composition.

30. The method of claim 29 wherein said dry free-flowing powder is formed by spray chilling.

31. The method of claim 29 wherein said dry free-flowing powder is formed by spray congealing.

32. The method of claim 29 wherein said dry free-flowing powder is formed by drum chilling.

33. The method of claim 29 wherein said dry free-flowing powder is formed by granulation.
